(19) United States
(12) Plant Patent Application Publication
(10) Pub. No.: US 2019/0090404 P1
(43) Pub. Date: Mar. 21, 2019

(54) Cannabis plant named 'EREZ'

(50) ;

(71) Applicant: TIKUN OLAM LTD, Tel Aviv (IL)

(72) Inventor: Ytzchak Cohen, Tel Aviv (IL)

(21) Appl. No.: 16/350,359

(22) Filed: Nov. 6, 2018

Related U.S. Application Data
(63) Continuation of application No. 14/757,040, filed on Nov. 12, 2015, now abandoned.
Application No. 14/757,040 is a continuation of application No. 14/193,197, filed on Feb. 28, 2014, now abandoned.
(60) Provisional application No. 61/768,564, filed on Feb. 25, 2013.

(51) Int. Cl. A01H 6/28

(52) U.S. Cl. PLT/263.1

(57) ABSTRACT

The disclosure relates to a new and distinct cultivar of Cannabis sativa plant named ‘Erez’, characterized by a high amount of Cannabidiol (CBD) (>16%) and a higher amount of Tetrahydrocannabinol (THC, ˜23%).

### BACKGROUND

The disclosure is directed to a new and distinct cultivar of Cannabis sativa plant named ‘Erez’, characterized by a high amount of Cannabidiol (CBD) and a high amount of Tetrahydrocannabinol (THC).

### SUMMARY

Provided herein is a new and distinct Cannabis sativa L ssp. indica plant named ‘Erez’, as illustrated and described herein, having a particularly high level of CBD of more than 16% and particularly low level of THC of less than 1%.

### BRIEF DESCRIPTION OF THE DRAWINGS

FIG. 1 illustrates a single fully grown plant of the variety;

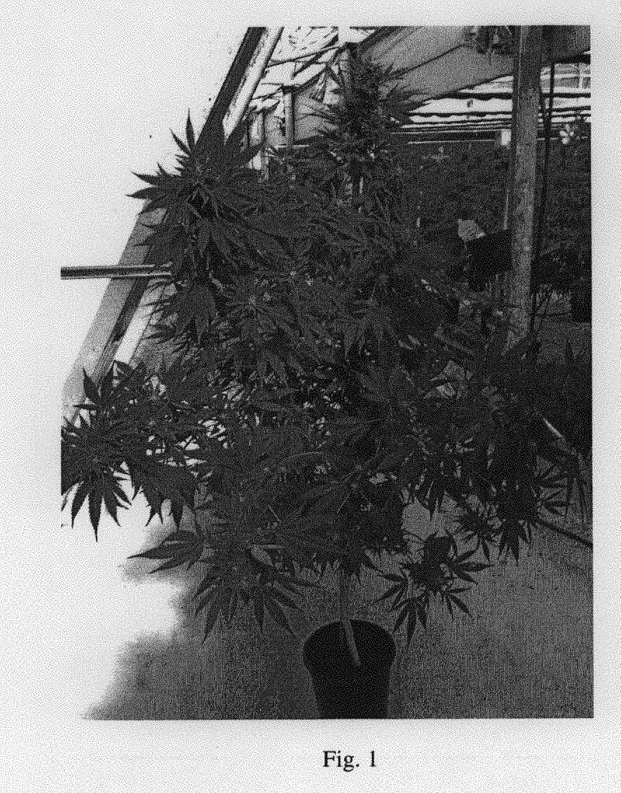

FIG. 2 illustrates a flowering plant in close-up;

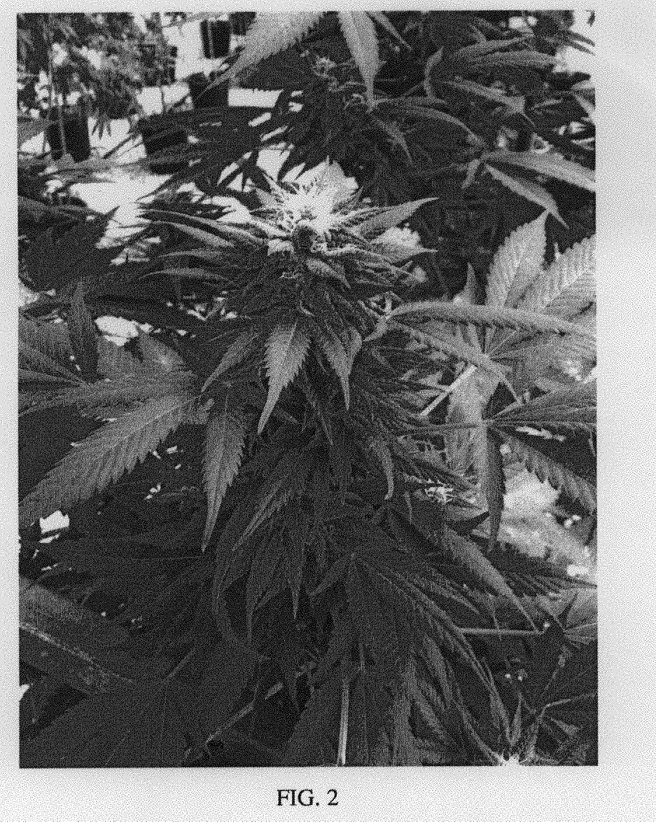

FIG. 3 illustrates a single leaf of the variety; and

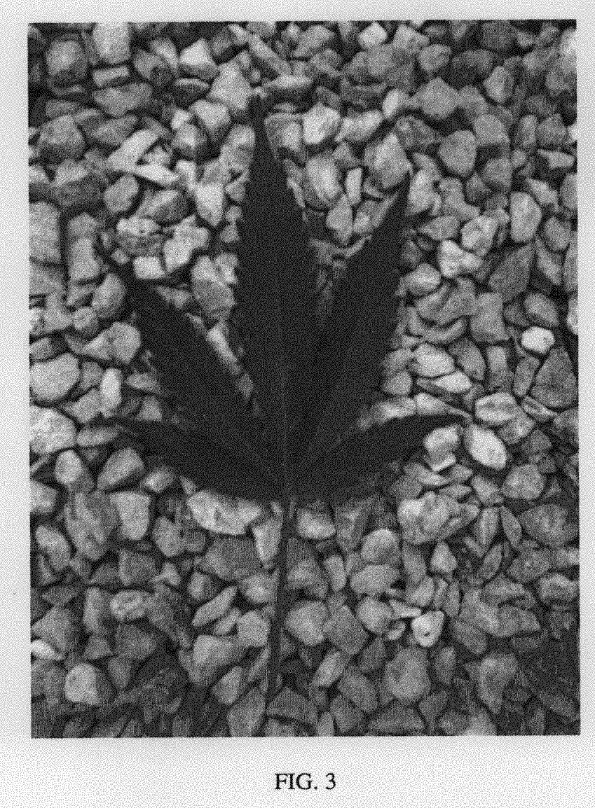

FIG. 4 illustrates a dried bud.

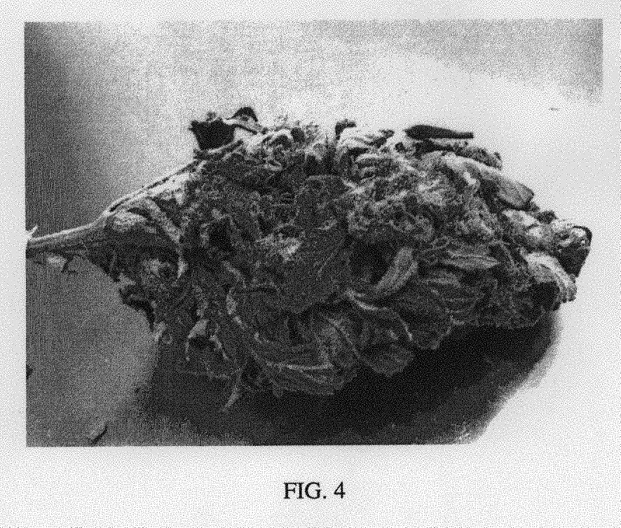

### DESCRIPTION

This invention relates to a new hybrid marijuana plant, Cannabis sativa L ssp. indica named Erez. The Erez is a medicinal variety of marijuana notable for its high proportion of THC to CBD.

The present plant was developed over 3 years through selective breeding from parents of unknown provenance in Birya, Israel. Propagate from seed, but large scale production generally occurs through asexual propagation using stem cuttings.

### PLANT

Exposed plant structure: This is an annual, dioecious plant. The natural height at 5 months old for indoor growth is 140 cm-165 cm, and for outdoor growth is 182 cm-215 cm. A detailed list of characteristics follows:
   Botanical classification.—Cannabis sativa L. ssp. indica.
   Parentage.—Unknown.
   Propagation.—Stem cuttings.
   Time to initiate roots in summer.—About 12 days.
   Plant description.—Annual, dioecious flowering shrub; multi-stemmed; vigorous; freely branching; removal of the terminal bud enhances lateral branch development.
   Root description.—Short taproot (less than 30 cm). Medium to fine, dense lateral roots; white in color, brownish when “potbound” (rootbound).
First year stems :
   Shape.—Round. Fine pubescence.
   First year stem strength.—Strong.
   First year stem color.—Young stem: 144C; older stems: 144A (The Royal Horticultural Society Colour Chart, 1995 Ed.).
   First year stem diameter.—Between 3 cm to 4.5 cm.
   First year trunk diameter.—Between 8 cm to 10 cm at the soil line.
   Stem strength.—Lateral stems are strong but benefit from being staked during flowering.
   Internodes length.—7 cm in the lower half and 4 cm in the upper half.
Foliage description:
   Texture (upper and lower surfaces).—Upper surface scabrid with non-visible stiff hairs; lower surface more or less densely pubescent, covered with sessile glands.
   Branch strength.—Strong.
   Branch description.—Branches are short, dense with short, broad leaflets.
   Leaf arrangement.—Palmately compound (digitate) leaves with 5 or 7 leaflets per leaf. The bottom two leaflets are angled upwards at about a 45 degree angle towards the middle leaflet.
   Venation pattern.—Palmately Compound (Digitate), with serrated leaflets. Each serration has a lateral vein extending to its tip from the central (primary) vein of the leaflet. From each lateral vein there is usually a single spur vein (sublateral vein) extending to the notch of each serration.
   Petiole length.—Average length of petiole of fan leaves 8 cm.
Vegetative bud description: The wet flower buds have many long white pistils, which become brown a week before harvest. The dried flower buds are a medium to dark green, with orange/brown pistils (hairs).
Flower description:
   Inflorescence description.—Cone — or head-like, dense cluster of false spikes with solitary flowers instead of cymes. Enclosed by glandular, beaked bracteole; Flowers are often paired. Petals are absent.
   Wet flour color.—Wet flower buds have many long white pistils, which become brown a week before harvest.
   Dried flower color.—Dried flower buds are a medium to dark green, with orange-brown hairs.
   Spike length.—Ranges between 5 cm and 25 cm.
   Pistils description position.—On buds, white and turning brown a week before harvest.
   Flower size.—About 1 cm in diameter and about 1.1 cm in height.
   Flowering time.—8 weeks.
Penduncles:
   Strength.—Strong, but can bend horizontally from weight of flower buds.
   Texture.—Moderately smooth, glabrous.
   Pedicels description.—Short, scabrid with sessile glands and visible hairs.

The fragrance is slightly sweet, having a fruity, ‘green”/‘fresh grass’, musky, medicinal smell. It is difficult to get this strain to produce male inflorescence

Seeds may be acquired through STS breeding. This involves inducing a female plant to become a male plant by altering its hormones using (in this case) silver nitrate and sodium thiosulfate. The male plant may then be used to pollinate a female of the same variety and collect the seeds. The relative proportion of male plants is medium/high.

The plant variety is similar to the Avidekel, also a Cannabis indica subspecies.

The inflorescences (flower buds) of the female plant are used for medical purposes.

This plant is very versatile. It can be used to treat a wide range of health disorders. It has many beneficial medicinal qualities. Some uses include:
Stimulant
Anti-inflammatory
Pain management
Digestive disorders
Sleep disorders
Anti-nausea and vomiting (increases appetite)
Tourette syndrome
Parkinson's disease
Spasms
Post-traumatic stress disorder (PTSD)

During the drying process, the buds tend to dry quicker than other varieties, and therefore require adequate relative humidity (>55%)

This variety prefers ‘soft water’; tap water should be filtered if it has an EC higher than 0.6 μS/cm.

The plants are generally grown with the aid of rooting hormone and synthetic pesticides.

| USDA Plants Growth Habit Code | FB |
|---|---|
| Vigor | 5/5 |
| Productivity | Good-500 g/m2 indoor, |
| | 250-400 g/plant outdoor |
| Male flowering timing | average |
| Flowering score | 7.5 |
| Branches | strong |
| Fruit | bloated |
| THC level | 23.0% |
| CBD level | 16.3% |

Permits are required for growth of medical marijuana in certain states in the US and in Israel.

1. A new and distinct Cannabis sativa L. ssp. indica plant named Erez, characterized by a high amount of Cannabidiol of greater than approximately 16% and a higher amount of THC of about 23%, as illustrated and described herein.
